(19) United States
(12) Patent Application Publication
(10) Pub. No.: US 2011/0066096 A1
(43) Pub. Date: Mar. 17, 2011

(54) DEVICE FOR TRACTION WOUND CLOSURE

(76) Inventor: Pal Svedman, Malmo (SE)

(21) Appl. No.: 12/557,868

(22) Filed: Sep. 11, 2009

(51) Int. Cl. A61F 13/02; A61M 1/00

(52) U.S. Cl. 602/53; 604/290

(57) ABSTRACT

Devices and methods aimed at shortening the time required for healing open wounds are disclosed. The tissues adjacent to wounds are expanded and/or stretched medially by means of an actuator which compressed by vacuum exerts traction on adjacent skin or tissues by means of an adhesively applied circumferent or transverse flange, thus reducing the size of the wound. The degree of vacuum, and traction, may be varied according to demand and applied continuously, cyclically or intermittently. Active treatment of the wound may be accomplished concomitantly by means of a conduit through the said actuator.

### FIELD OF THE INVENTION

The instant invention relates to wound healing and more particularly, but not by way of limitation, devices and methods for stretching and expanding adjacent tissues medially to cover a wound.

### BACKGROUND

Open clinically significant wounds heal by contraction, a slow process which includes scar contraction and re-epithelialization and leaves scar and usually functional and aesthetic deformity. Disfigurement and functional disturbance is frequent. Surgical treatment, which can prevent, break or ameliorate the course, may be delayed or contraindicated for various reasons, or may just not be an available option. As a consequence, deformity and scarring are problems to be addressed.

Mechanical forces play an important part for tissue development and function through effects induced on the cellular and extracellular level. When traction is applied protractedly to the skin from a surgically implanted, outwardly expanding balloon (i.e. a tissue expander), an area of the overlying skin and subcutis increases by formation of new tissues, and can be used surgically for covering an adjacent open wound. During the expansion, the epidermal thickness, i.e. the number of cells, increases, and that of dermis and subcutis decreases. Blood flow in the expanded tissue increases, and sensitivity remains intact. When skin traction is performed acutely, markedly higher stretching forces have to be applied, and the degree of skin lengthening is the result of mechanical stretching rather than formation of new skin.

Surgical devices have been disclosed which use skin expansion or acute stretching to close open wounds of surgical or non-surgical etiology. Technically, pins or hooks are fastened near the wound's edge at opposing sides, usually through several fixture points in the fibrous dermal layer of the skin. Medial traction is accomplished by means of sutures, rubber bands, plastic straps or screws. By these means, skin is expanded or stretched medially until wound closure can be achieved. Disadvantageously, the traction force becomes reduced over time if the sutures of bands are not readjusted at intervals. Also, access for effectively treating the underlying wound becomes restricted. Neither do such approaches allow centripetal traction nor predetermined variation of traction to maximize tissue gain.

In abdominal compartment syndrome of differing etiology increased intraabdominal pressure requires pressure decompression. The abdomen is opened by means of a long medial incision through the anterior abdominal wall which includes fascia and peritoneum. This makes the abdominal wall including fascia contract laterally to such a degree that surgical closure can be accomplished only after the tissue has stepwisely been stretched until its normal width has been regained. Technically, this is accomplished by suturing a non-yielding perforated polymer mesh circumferentially to the exposed edges of the abdominal wall fascia under traction, and repeating the procedure at 2 to 3 day intervals until the abdominal wall length has been regained. At this point, the mesh is removed and the wound resutured in layers. Throughout, the wound is usually exposed to negative pressure treatment, which also allows removal of excess abdominal fluid contributing to the distension.

The rate of formation of new tissue relates to the magnitude and duration of the applied traction force, and to the width of tissue (skin) exposed to traction of a given magnitude. The relationship between these parameters has not been evaluated systematically, but loading an undefined width with 0.5 kg-3.0 kg (6.9N-29N) is reportedly a reproducible means to produce such skin expansion in adult patients. Using broad straps adhered to the skin, this range of loading is used also to disengage fractures or dislocations. In order to lengthen skin acutely, higher weights are required.

Treatment of wounds by means of negative pressure applied through a contractile open cell foam dressing includes tissue traction. Both the wound and the adjacent skin become exposed to medial pull as the dressing contracts during application of suction. The degree of pull is limited by friction between the foam cells and onlying polymer film, between onlying cells, and between said cells and wound bed. A limitation is that the level of negative pressure cannot be increased beyond the upper pressure range recommended for wound treatment.

### SUMMARY OF INVENTION

It is a general object to improve wound healing.

It is a further object to provide a device for wound healing.

A further object is to provide a device and method for improved traction in treating wound healing.

An object is to improve the treatment of open major wounds to prevent disfigurement and functional disturbance.

Such outcomes may be counteracted by means of the present invention, which by providing new means and methods for enlongating tissue in a controlled way allows intact skin and subcutaneous tissue to be moved medially to markedly reduce the size of the wound, and eventually achieve non-surgical wound closure with innervated composite tissue in kind. As for the present invention, the primary field of use is for achieving wound closure by protracted traction, although closure through acute traction may also be feasible. In either application, the distribution of traction force evenly at the skin surface level (rather than concentrated to points of dermal penetration) prevents local tissue compression and may thus avoid skin necrosis.

The invention may also be used in patients with open abdomen to stretch contracted abdominal wall.

Use of the invention may typically be indicated 1) when reconstructive surgery cannot be undertaken because of the patient's condition, 2) when for various reasons such surgery has to be delayed, and 3) when surgical treatment is unavailable. Its use is contraindicated when the wound or the adjacent skin is damaged or infected, and when the skin has poor blood circulation or is inflicted by disease engaging the dermo-epidermal junction.

More specifically, traction to the skin adjacent to a wound is accomplished according to the invention by means of an actuator placed over the wound and adhered to the surrounding skin by an adhesively applied flange which may be circular or divided into two parts extending laterally. The said actuator interior consists of elastic, compressible open cell material, and is airtightly connected to a suction pump by means of a tube according to the state of the art. When exposed to negative pressure, the actuator contracts medially and the force created by suction exerts medial traction on said flange(s) and underlying skin and/or tissue. Lubricant may be added to the cell material to reduce interior friction and facilitate contraction. The maximal pressure range may extend from 50 mmHg to 1000 mmHg below atmospheric pressure, with clinical range between 100 mmHg and 650 mmHg below atmospheric pressure. The amount of medial pull is dependent upon the level of the applied negative pressure, the compressability of the cell material and the resistance to traction offered by the tissues underlying the said flange(s). Under in vitro conditions, the medial contraction of an actuator has reached 90 percent. The adhesive flange(s) may be inelastic or elastic and yielding to stretch to an extent that variably accomodates the increase in length as the underlying skin becomes stretched medially. When the flange is elastic, the traction force may become distributed over a relatively wide area of the adhered skin, minimizing the risk of tangential shear within the skin. The said skin flange(s) may or may not be fitted with an inner, circumferent rim free from adhesive, and should preferably be transparent to allow visual assessment of the underlying skin with respect to viability. The flange film may finally contain pores which allow evaporation of water, which may reduce skin adhesivity. Clinically, the devices according to the invention may be changed from every three to six days.

The undersurface of the adhered actuator may cover the wound only partially, leaving access for applying a wound dressing from the sides. This dressing may include active substances distributed in fiber, open cell or gel material, or it may constitute an occlusively applied typically open cell, pore or fiber dressing with access port(s) used for negative pressure wound treatment, eventually combined with fluid supply. To provide access also when the device according to the invention blocks access to the wound, the actuator may be fitted with at least one conduit, typically extending between its exterior and interior side. Said conduit allows room for at least one flexible tube. As described above, said tube(s) can for instance accomodate negative pressure wound treatment with or without concomitant supply of treatment fluid to an underlying open cell pore or fiber dressing according to the state of the art.

The actuator according to the invention may be used as roof in a wound treatment chamber during on-going actuator traction. In this application the external aperture of said actuator conduit is fitted with resealable port means. Such treatment may for instance include delivery of analgesics and antibiotics. The chamber may furthermore be used as growth chamber and supplied with growth factors, growth media, genes and cells, including stem cells and fetal cells. In both applications, the chamber may contain a biological or synthetic matrix acting as a scaffold for cell growth.

The pump used for compressing the actuator by suction should include a gauge for measuring negative pressure, means to adjust and maintain the pressure according to clinical demand, and an alarm to warn of air leak in the system, wherein all means can be state of the art. The pump means may be controlled by computer technology, eventually by telemetry or manually. The pumps may be portable. A manual or syringe pump fitted with manometer may be used under special circumstances.

The controlled continuous, intermittent or cyclic application of skin traction according to the invention may enhance blood flow and stimulate tissue expansion, applications based on known regenerative effects on cell and tissue function, and may find use for testing a range of stretch stimuli with regard to further regenerative effects.

When suction therapy is administered through an underlying occlusively applied open cell, pore or fiber dressing, a preferred option may be to use a double suction pump connected via separate tubes to actuator and dressing respectively. Suction may be applied to actuator and underlying dressing simultaneously, or to the dressing only after a slight delay. The reason for the latter is that activation of the actuator is expected to constitute the main wound contracting force. The combined negative pressure of actuator and dressing should remain well below that causing stretch-related damage of the adjacent skin.

In a special application, one special application of the invention is used in connection with open abdomen, i.e. extensive wounds penetrating through the thickness of the abdominal wall, often combined with abdominal compartment syndrome. In this application the device according to the invention is used within the wound. The flange of the device is typically connected by suture to the abdominal fascia edges rather than adhered to the skin surface. The flange consists of a perforated mesh whose pores permit excess abdominal fluid to be drained from the underlying abdominal cavity.

One embodiment of the invention is directed to a device for applying traction to tissues adjacent to a wound. The device includes (a) a contractible actuator to be positioned within a wound; (b) a cover, which can be flexible, configured to enclose the actuator and configured to maintain reduced pressure and degree of contraction therein; (c) a flange extending laterally from an actuator base; (d) a reduced pressure supply means for connection to a source of suction, the reduced pressure means cooperating with the cover to supply the reduced pressure within the the actuator; and (e) an actuator means contracted by suction cooperating with the flange to produce medial traction of adjacent fascia and abdominal wall tissues, and reduce the size of the wound.

By virtue of the instant invention, there is less chance for deformity and scarring to occur.

### BRIEF DESCRIPTION OF THE DRAWINGS

Embodiments of the present invention will now be described by way of example only and with reference to the accompanying drawings of which:

FIG. 1 shows a schematic perspective view of a device for circumferent traction closure according to one embodiment of the invention with a partial reveal.

FIG. 2 shows a schematic cross-section of an embodiment with a partial reveal before being shaped and assembled.

FIG. 3 shows a schematic frontal view according to FIG. 2.

FIG. 4 shows a schematic cross-section of an embodiment applied over a wound.

FIG. 5 shows the same general embodiment after applying traction by means of suction.

FIG. 6 shows a schematic view of another embodiment of the invention used for achieving transverse traction closure.

FIG. 7 shows schematic cross-section of a pre-assembly embodiment fitted with a conduit.

FIG. 8 shows a schematic frontal view of the embodiment according to FIG. 7.

FIG. 9 shows a schematic perspective view of an embodiment with a double pump with suction tubes, one tube passing through a conduit in the device.

FIG. 10 shows a schematic cross-section of an embodiment combining traction to the skin around the wound with negative pressure treatment (which includes an element of traction) of an underlying occlusively applied open cell or pore dressing.

FIG. 11 shows a schematic cross-section of another embodiment with conduit during ongoing suction. Negative pressure treatment combined with restricted fluid supply is concomitantly applied via the said conduit to an underlying open pore dressing applied to the wound.

FIG. 12 shows a cross-section of a final embodiment comprising a wound treatment chamber underlying an actuator with conduit acting as a roof. The chamber, filled with therapeutic fluid, can be accessed by removing a resealable plug from said conduit.

FIG. 13 shows an embodiment of a pre-fabricated actuator fitted with an open polymer mesh flange. Here, it is used in the depth of the wound as a bridge between the edges of the abdominal fascia on either side of the wound. By means of this device the width and volume of the opening in the abdomen may be controlled, and when the measured intraabdominal pressure permits, traction can be applied over a period of time to stretch contracted tissues, permitting eventually surgical abdominal closure. The flange of the actuator is sutured to the fibrous fascia layer of the abdomen. Intra-abdominal fluid may pass from the abdominal cavity, through the said polymer meshes of the flange, and into a suitable overlying wound treatment device, typically a negative pressure wound therapy device.

FIGS. 14-15 exemplifies clinical use of the prefabricated embodiment of the invention shown in FIG. 13.

FIGS. 16-19 exemplify clinical use of a self-assembled embodiment according to the invention shown in FIG. 13.

FIG. 20 shows a computerized state of the art suction pump which has been combined with a unit for measure the intraabdominal pressure.

FIG. 21 shows a cross sectional for part of the invention.

### DETAILED DESCRIPTION

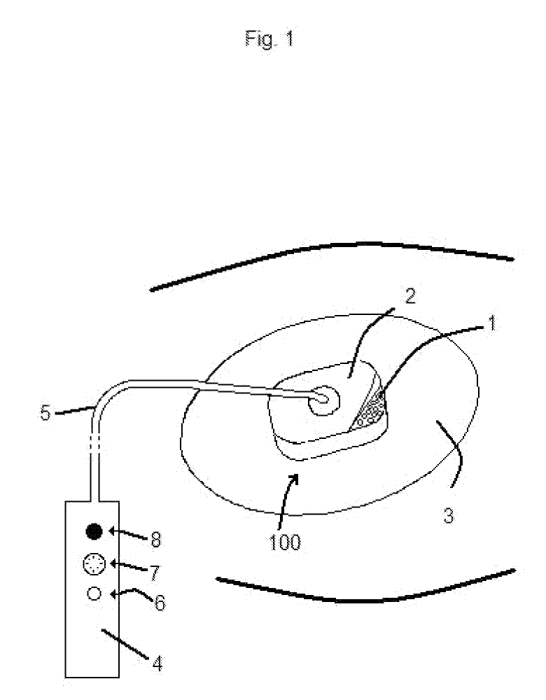

Referring now to the drawings, FIG. 1 shows a schematic clinical view of one skin traction device according to the invention before activation. The actuator 100 may preferably consist of compressible open cell or porous polymer material 1, for instance polyester or polyurethane (Minnesota Mining and Manufacturing, St. Paul, Minn. 55144) or alternatively polymer fiber material according to the state of the art. The compressible material 1 is enclosed on all sides by a flexible polymer film sheet 2 consisting for instance of polyester, polyurethane or latex. The sheet 2, which may or may not be gas impermeable and transparent, extends into an adhesive film flange 3 preferably of similar polymer material. A state of the art suction pump 4 can be connected to the interior of the actuator 100 by means of flexible polymer tube 5 and adhesive flange 55, wherein the tube 5 can include an inner longitudinal ridge to prevent occlusion during kinking.

The pump 4 is fitted with hardware and software 6 for adjusting the level of negative pressure, and a gauge with display 7 indicating the said level visually. State of the art computerized processor with associated hardware and software 6 for varying the level of negative pressure in actuator 100 automatically according to pre-set conditions can be included, with cycles as short as 30 seconds. State of the art computerized alarm hardware/software 8 for detecting and warning of eventual air-leak in the system are preferably included.

The device according to the invention is typically fitted to the wound bedside. The open cell pad 1 and the polymer sheets 2 may either be cut separately to the size and shape of the required to form actuator 100 including its covering layer 2 and connected flange 3, or a partly pre-assembled device may be used which facilitates the procedure.

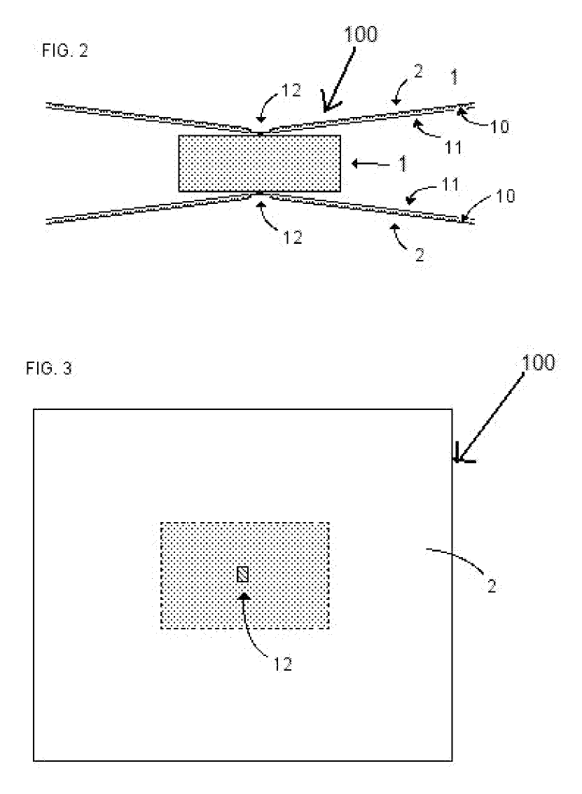

FIG. 2 shows a cross-section of a skin traction device according to FIG. 1 before being cut and assembled to fit the preferred shape and size relative to the wound and adjacent skin. The compressible open cell material unit 1 and the polymer film sheets 2 are shown, the latter with the adhesive side 10 protected by a releasable layer of polymer film 11. The polymer film sheets 2 have been connected to the surface of open cell material 1 at adhesion points 12 on either side. FIG. 3 shows a frontal view of the partly assembled actuator 100 according to FIG. 2.

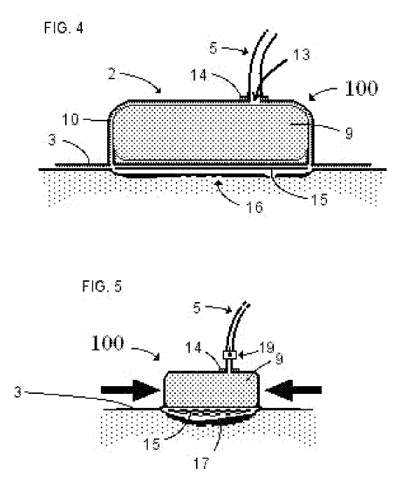

FIG. 4 shows a cross-section of a device according to the invention in position over open wound 16. The polymer film sheet 2 encloses the open cell material 1 with its adhesive side 10 facing inwards onto the cell material 1, and forming circumferent adhesive flange 3. A flexible polymer suction tube 5 is connected to hole 13 in polymer sheet 2 by means of polymer suction port 14 which is adhesively applied to polymer sheet 2 adjacent to hole 13 according to the state of the art. Suction port 13 may or may not form an integrated part of suction tube 5. A small volume of lubricant, for instance medical grade oil, may be injected through the port 14 prior to applying suction and through compression repeatedly provides for the oil to spread. A wound dressing 15 is positioned within wound cavity 17.

FIG. 5 shows the effect of applying continuous suction to tube 5 of the device shown in FIG. 4. The vacuum established in actuator 100 exerts traction on flange 3, the direction indicated by the arrows. The dressing 15 has been moved medially within wound cavity 17. Actuator 100 is for practical purposes air-tight, and suction port 14 or tube 5 may or may not be connected with an airtight valve 19 according to the state of the art. This valve 19 may allow vacuum to be maintained for a shorter or longer period without ongoing suction pump action.

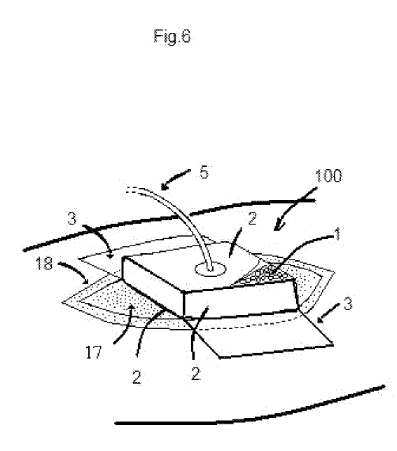

FIG. 6 exemplifies a device according to the invention applied transversely across a cavity wound 17. A polymer film sheet 18 is adhesively applied to the skin at the edges of the wound 16. The actuator 100 with its polymer film cover 2 is free from non-adhesive surface of the film 18 covering wound 16. Flanges 3 are adhesive only lateral to film 18. In this schematic clinical example, suction has not yet been applied through tube 5.

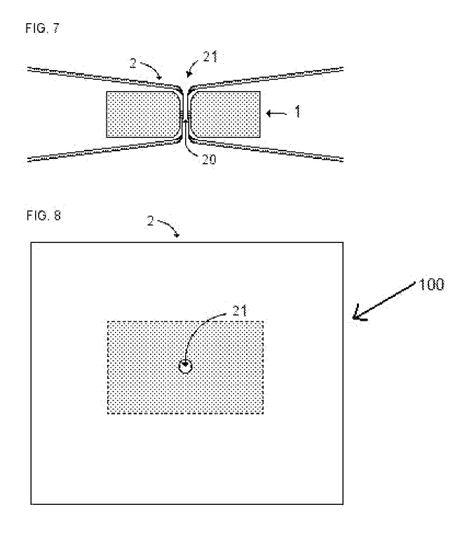

FIG. 7 shows another example of a cross-section of pre-assembled actuator parts before being cut to the preferred shape and size relative to the wound and adjacent skin. The parts are the same as described in FIG. 2 except for passageway 21 with walls covered by polymer film 2. Passageway 21 may be fitted with adhesion points 20 to open cell material 1 to keep the polymer film 2 in place. Alternatively points 20 may be substituted with an elastic or rigid adhesive ring of polymer material. FIG. 8 shows a frontal view of the partly assembled actuator 100 according to FIG. 7.

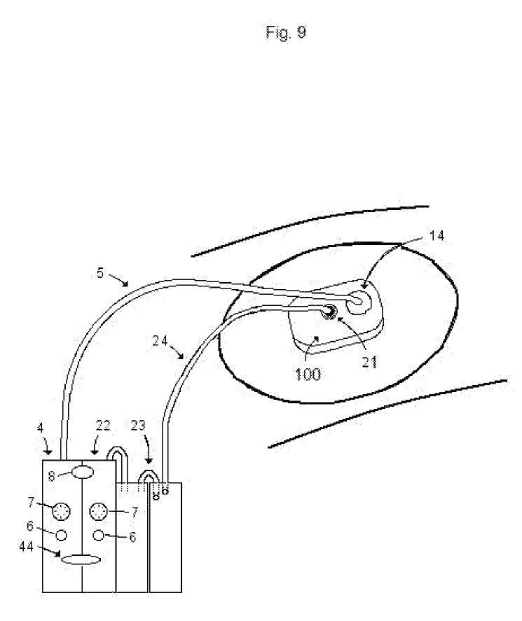

FIG. 9 shows a schematic clinical view of a traction device according to the invention in which actuator 100 fitted with conduit 19 (see FIGS. 7 and 8) is in use on a patient. A double suction pump 4, 22 is used. The traction system, comprising actuator 100, port 14, tube 5 and pump 4 corresponds to that described in FIG. 1. In addition, passageway 21 accomodates tube 24 which allows suction from pump 22 to be applied to an underlying dressing. Pump 22 may be fitted with an overflow control 23. Computer electronic hardware and software 44 according to the state of the art allows synchronous or separate operation of negative pressure time courses from pumps 4 and 22 to be predefined. Negative pressures may for instance be applied to actuator 100 continuously, cyclically or sequentially, the latter with minimal delay before actuating vacuum to the underlying dressing. The maximal level of vacuum administered to the dressing is limited to the upper therapeutic range for wounds. The negative pressures in the two pump channels may preferably be synchronized. To maximize the tissue traction the two pumps 4, 22 may be activated simultaneously or one after the other as required. Alarm hardware and software 8 indicate technical malfunction, and may include means for telemetric warning to personnel on duty according to the state of the art.

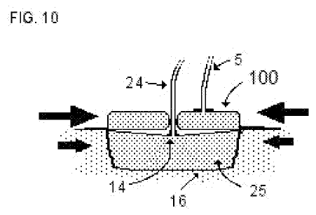

FIG. 10 shows a schematic cross-section of a negative pressure operated device according to the invention used in combination with negative pressure treatment of the underlying wound through open cell dressing 25. The negative pressure and hence medial traction in the actuator 100 and wound dressing 25 layers may be independently controlled and varied, see under FIG. 9 for means and scheme involved. The compressibility of the open cell material of wound dressing 25 may exceed that of actuator 100 to compensate for higher negative pressure and thus larger volume reducing effect caused by the latter. The arrows indicate that, in this example, the negative pressure applied to actuator 100 produces stronger traction than that of negative pressure treatment applied through the underlying open cell dressing 25. The total skin and subcutaneous tissue traction force constitutes the sum of the traction forces applied to actuator 100 and occlusive wound dressing 25. By for instance varying the level of negative pressure in the two layers cyclically, also by changing the amplitude of the pressure difference over time, a standardized, defined combined stretching and relaxing stimulus can be produced, which may tentatively influence blood flow and tissue growth favorably. To maximize open pore material responsiveness to such change in negative pressure, the dead volume of tubes 5 and 24 may be minimized.

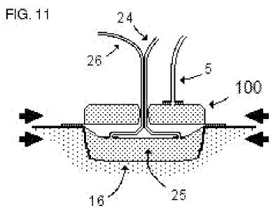

FIG. 11 shows a device according to the invention as described in FIG. 9 in which an underlying open cell dressing 25 receives suction through tube 24 and supply of treatment fluid through tube 26. As shown by the arrows, actuator 100 and dressing 25 are in this example exposed to the same level of traction.

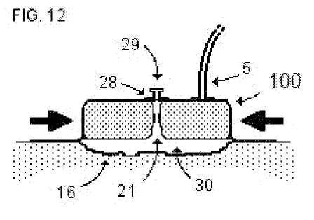

FIG. 12 shows another device according to the invention which has been adhered to the skin adjacent to a wound before applying suction to tube 5, thus activating traction as indicated by the arrows. The actuator 100 comprises passageway 21 which has been fitted at its external side with an adhesively applied port 28. Port 28, fitted with lid 29, is resealable. An occluded chamber 30 is thus created which may be used for delivering active treatment to the wound, including use as a growth chamber.

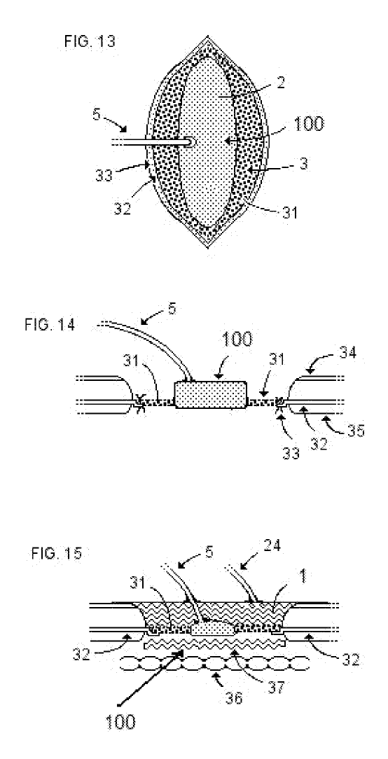

FIG. 13 shows a device according to the invention used for stretching the abdominal fascia in abdominal wounds of patients treated for abdominal compartment syndrome. The device may be prefabricated, and different sizes comprising preferably oblong actuators may be required to provide a necessary size range. A schematic perspectivic view of the device in place in an abdominal wound before applying suction to tube 5 is shown. Actuator 100 is completely covered by said clinically air-tight polymer film 2. The outer rim of flange 3, in this case constituted by open polymer mesh 31, is fixed to the inner rim of the abdominal fascia 32 by means of sutures 33. Alternatively, the mesh 31 may be substituted by open pore, cell or fiber material, all typically open to passage of body fluids. Clinically, the device needs to be complemented with outer and inner dressing components, see below.

FIG. 14 shows a schematic cross section of the device shown in FIG. 13 in place in the abdominal wound, demonstrating the depth at which the mesh 31 is sutured to the fascia 32. The outer epidermal layer of the skin 34 and the inner peritoneal layer 35 of the abdominal cavity are indicated.

FIG. 15 shows a further schematic cross-sectional clinical view. Externally, a negative pressure dressing with open cells 1 has been occlusively applied and is externally connected with suction tube 24. Underneath, the traction treatment prepared according to FIGS. 13 and 14 has been activated by applying vacuum to actuator 100 through tube 5, and the fascia 32 and the abdominal wall tissues on either side have become extended medially by traction. In between the intra-abdominal tissues 36 and the device according to the invention, another open cell dressing 37 has been applied to avoid compression and facilitate evacuation of intraabdominal fluid through the meshes 31 of flange 3. The liquid is finally drained by suction through tube 24. A manual method of shaping and assembling a device according to FIGS. 13-14 is indicated in the following.

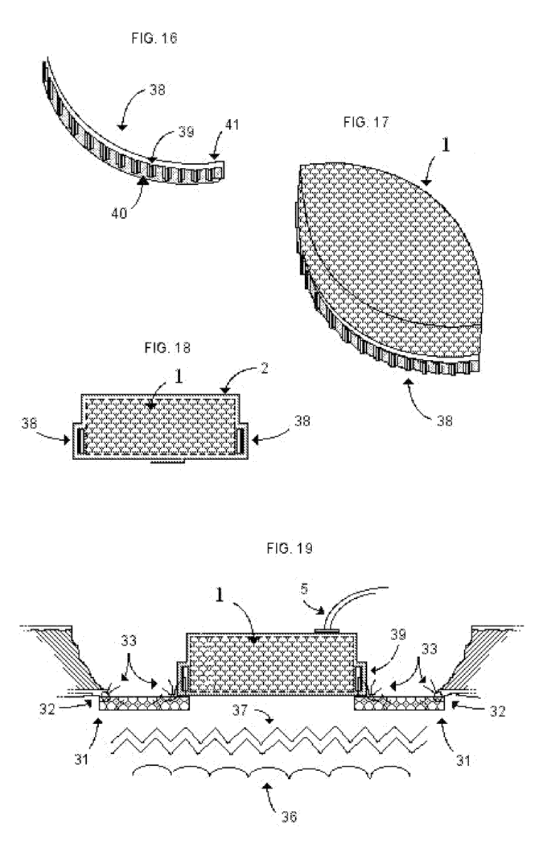

FIG. 16 shows schematically a flexible, compressible securization band 38 to be used as part of actuator 100. Band 38 is fitted with a series of protruding ridges 39 covered by adhesive gel 40 on the intended outside, and with adhesive sheet 41 on the inside.

FIG. 17 shows band 38 adhered in place circumferentially along the base of the open cell unit 1 of actuator 100.

FIG. 18 shows a cross-section of a preassembled actuator 100. An adhesive polymer film cover 2 is applied airtightly to open cell material unit 1 with securization bands 39 in place inside the adhesive surface of cover film 2. The ridges are used as fixation points when suturing actuator 1 to open mesh 31.

FIG. 19 shows a cross-section of actuator 100 according to FIG. 17 in place in the abdominal wound. The protruding ridges 39 have been fixed to the inner edges of flanges 3 by means of sutures 33, and the outer edges of flange 3 have in turn been sutured to the abdominal fascia 32 on either side of the wound. Suction is applied to actuator 1 via tube 5. A pliable open pore dressing 37 has been interposed between actuator 1 and the abdominal tissues 36 to prevent injury and facilitate outwards drainage from the inside of the abdomen.

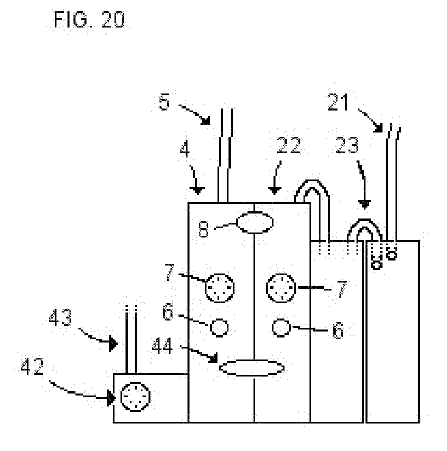

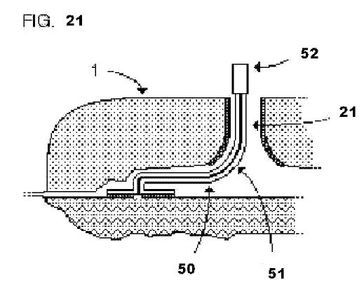

FIG. 20 shows a schematic perspective view of a double suction pump according to FIG. 9 comprising also tube 43 connecting distally with a catheter for measuring the intra-abdominal pressure according to the state of the art, and proximally with manometer 42. This part of the device is used for continuous or intermittent measurement of the intra-abdominal pressure. The suction pump/computer means according to FIG. 9 may comprise means for monitoring the said intra-abdominal pressure continuously of intermittently, as well as means whereby the pressure in actuator 100 can be automatically reduced if the intra-abdominal pressure exceeds a certain level. A built-in alarm function warning of such increased intra-abdominal pressure may also be included

FIG. 21 shows a distal connection tube 50 intended for fluid supply or suction drainage through passageway 21 in accordance with FIG. 11. To avoid kinking in the limited space available, its flexible polymer tube wall 51 is thickened and may comprise an internal longitudinal ridge. Tube 50 is proximally fitted with a Luer connector 52, or alternatively with a tubular, elastic connector.

It will be seen from the objects above and those apparent from the description, that modifications, derivations and improvements can be made without departing from the invention and it is intended that all matter contained in the above description and shown in the accompanying drawings shall be interpreted as illustrative and not in a limiting sense with respect to the claims appended hereto.

I claim:

1. A device for applying traction to skin adjacent to a wound, comprising:
(a) a contractible actuator configured to be positioned over a wound and having a base;
(b) a flexible cover configured to enclose said actuator on all sides and configured to maintain reduced pressure and degree of contraction therein;
(c) a flange extending laterally from said actuator base;
(d) means for sealing the said flange to the adjacent skin;
(e) reduced pressure supply means for connecting to a source of suction, said reduced pressure means cooperating with said cover to supply said reduced pressure within said actuator; and
(f) actuator means for contracting by suction cooperating with said flange to produce traction of adjacent skin and underlying tissues, and reduce size of the wound.

2. A device according to claim 1, wherein said actuator comprises one of an open cell, pore and fiber material.

3. A device according to claim 1, wherein said actuator includes an elastic member.

4. A device according to claim 1, wherein said seal means includes an adhesive material to secure said actuator to tissue surrounding the wound.

5. A device according to claim 1, wherein said flange includes a circumferent extension of said actuator base.

6. A device according to claim 1, wherein said flange includes an extension from a side of the actuator.

7. A device according to claim 1, wherein said actuator comprises a passageway between an exterior side and interior side thereof for administering treatment to said wound.

8. A device for treating a wound by applying traction to adjacent skin and tissues comprising:
(a) a contractible actuator configured to be positioned over a wound and having a base;
(b) a cover adapted to fully enclose the open cell material of said actuator, and maintain reduced pressure and degree of actuator contraction;
(c) a flange extending laterally from said actuator base exerting continuous medial traction on skin and underlying tissues adjacent to the wound;
(d) a seal for sealing said flange to tissues surrounding the wound;
(e) a reduced pressure supply for connecting said actuator to a source of suction by a tube having one of a port and valve; and
(f) a passageway through said actuator for access to underlying dressings placed on the wound.

9. A device for administering traction to the skin adjacent to a wound comprising:
(a) a vacuum system producing reduced pressure, wherein said vacuum system includes tubing and is adapted to maintain reduced pressure having a predetermined volume within a member in a manner to cause medial traction of the wound;
(b) a valve connected to said tubing; and
(c) means for intermittently applying reduced pressure through said valve.

10. A device according to claim 9, wherein said vacuum system includes a manual suction pump to provide reduced pressure.

11. A device according to claim 9, wherein said vacuum system includes a syringe to provide reduced pressure.

12. A device according to claim 9, wherein said vacuum system includes a an electrically operated pump to provide reduced pressure.

13. A device according to claim 9, wherein said vacuum system includes an actuator and a suction-dependent open pore dressing placed underneath said actuator and accessed through a passageway and operates said reduced pressure for said actuator and said dressing in one of a simultaneous and concurrent manner.

14. A device according to claim 9, wherein said vacuum system includes an actuator and a suction-dependent open pore dressing placed underneath said actuator and accessed through a passageway and operates to cause reduced pressure first for said actuator and second for said suction-dependent open pore dressing.

15. A vacuum system according to claim 8 wherein the reduced pressure is in a range between 50 mmHg below atmospheric pressure to 1000 mm Hg below atmospheric pressure.

16. A vacuum system according to claim 8 wherein the reduced pressure is in a range between 100 mmHg and 650 mm Hg below atmospheric pressure.

17. A method for treating a wound by traction, wherein the application comprises the steps of:
(a) placing an actuator compressible by suction, over the wound;
(b) sealing a flange of said actuator to adjacent tissue surrounding the wound;
(c) applying a treatment to an underlying wound; and
(d) actuating said actuator for producing the reduced pressure to achieve traction on the adjacent tissue and contraction of the wound.

18. The method for treating a wound by traction of claim 17, wherein there is a space provided between said actuator and the wound for tissue regeneration.

19. The method for treating a wound by traction of claim 18, which includes introducing fluid therapy into the space.

20. The method for treating a wound by traction of claim 17, wherein the step
(d) is characterized to actuate said actuator in one of an intermittent and a continuous manner.

21. The method for treating a wound by traction of claim 17, which includes repeating said steps until the wound is healed.

22. A device for applying traction to tissues adjacent to a wound, comprising:
(a) a contractible actuator to be positioned within a wound;
(b) a cover configured to enclose the actuator and configured to maintain reduced pressure and degree of contraction therein;
(c) a flange extending laterally from an actuator base;
(d) a reduced pressure supply means for connection to a source of suction, said reduced pressure means cooperating with said cover to supply said reduced pressure only within the said actuator; and
(e) an actuator means contracted by suction cooperating with said flange to produce medial traction of adjacent fascia and abdominal wall tissues, and reduce the size of the wound.

23. The device for applying traction to tissues adjacent to a wound of claim 22, wherein said flange includes a mesh with open pores.

24. A device according to claim 22, wherein said flange comprises one of an elastic and inelastic material.

25. A device according to claim 22, which includes a manometer and computer each operably associated with said actuator and reduced pressure supply means for measuring intraabdominal pressure.

26. A method according to claims 22, which provides one of continuous and intermittent measurement of abdominal pressure during reduced pressure wound treatment and providing computer controlled reduction of the reduced pressure in the actuator if intraabdominal pressure exceeds a predetermined limit.
